(19) United States
(12) Patent Application Publication
(10) Pub. No.: US 2012/0183554 A1
(43) Pub. Date: Jul. 19, 2012

(54) Biomarker

(76) Inventors: Richard Morgan, Guildford (GB); Hardev S. Pandha, Guildford (GB)

(21) Appl. No.: 13/383,399

(22) PCT Filed: Jul. 13, 2010
(86) PCT No.: PCT/GB10/01333

§ 371 Date: Mar. 26, 2012

(30) Foreign Application Priority Data
Jul. 13, 2009 (GB) 0912190.6

(51) Int. Cl. A61K 39/395; C07K 5/10; C07K 7/06; A61K 31/7088; C07K 16/18; G01N 33/566; A61K 38/17; C07K 14/47; A61P 35/00; A61P 37/04; G01N 21/64; C07H 21/04; A61K 39/00

(52) U.S. Cl. 424/139.1; 536/23.5; 530/330; 530/329; 530/328; 514/44 R; 424/178.1; 530/387.9; 424/185.1; 514/19.3; 530/350; 435/6.12; 435/7.1

(57) ABSTRACT

Described are gastrointestinal cancer specific biomarkers comprising the nucleic acid sequence of the Engrailed-2 (EN2) gene or the amino acid sequence of the encoded EN2 protein. Also described are uses of the biomarkers in the treatment, diagnosis, monitoring and imaging of gastrointestinal cancer.

The present application relates to biomarkers, in particular to biomarkers for gastrointestinal cancer.

Gastrointestinal cancer is a group of cancers that affect tissues and organs of the gastrointestinal tract. Examples of gastrointestinal cancer include oesophageal cancer, gall bladder cancer, stomach cancer (gastric cancer), liver cancer, pancreatic cancer, bile duct cancer, small intestine cancer, colorectal cancer and anal cancer. Colorectal cancer includes colon cancer and rectal cancer.

Some form of gastrointestinal cancer is newly diagnosed in more than 250,000 patients annually in the United States.

Despite advances in technology, gastrointestinal cancer remains difficult to treat.

### SUMMARY OF THE INVENTION

According to one aspect of the present invention, there is provided a gastrointestinal cancer specific biomarker, the biomarker comprising:—

(i) a nucleic acid sequence comprising SEQ ID NO:1, or a fragment or variant thereof, or a nucleic acid molecule which comprises said nucleic acid sequence; or

(ii) an amino acid sequence comprising SEQ ID NO:2, or a fragment or variant thereof, or an amino acid molecule which comprises said amino acid sequence.

In this respect, SEQ ID NO:1 corresponds to the nucleic acid sequence of the Engrailed-2 (EN2) gene (GenBank reference number NM_{—}001427) and SEQ ID NO:2 corresponds to the EN2 protein encoded thereby (NCBI accession number P19622, gi21903415).

Preferably, the gastrointestinal cancer is selected from oesophageal cancer, gall bladder cancer, stomach cancer, liver cancer, pancreatic cancer, bile duct cancer, small intestine cancer, colorectal cancer and anal cancer. Preferably, the colorectal cancer is selected from colon cancer and rectal cancer.

Surprisingly, it has been found that the EN2 gene is significantly up-regulated in gastrointestinal cancer.

The EN2 gene encodes a homeodomain-containing transcription factor that has a number of important functions in early development including axonal guidance and boundary formation (reviewed in Morgan R, (2006). Engrailed: Complexity and economy of a multi-functional transcription factor. FEBS letters 580, 2531-2533, which is incorporated herein by reference in its entirety). Its NCBI/GenBank reference number is NM_{—}001427. It has previously been reported to act as an oncogene in breast cancer, although no diagnostic significance has been attributed to it (Martin, N. L., Saba-El-Leil, M. K., Sadekova, S., Meloche, S. and Sauvageau, G. (2005) EN-2 is a candidate oncogene in human breast cancer. Oncogene 24, 6890-6901, which is incorporated herein by reference in its entirety). The EN2 gene product is a 33 kDa protein (EN2).

Preferably, the fragments or variants thereof comprise:—

(i) a nucleic acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% nucleic acid sequence identity with SEQ ID NO:1, a nucleic acid sequence that is hybridizable thereto under stringent conditions, and/or a nucleic acid sequence that is complementary thereto;

(ii) an amino acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% amino acid sequence identity with SEQ ID NO:2, or

(iii) an amino acid sequence encoded by a nucleic acid sequence of (i).

Put another way, in accordance with part (iii) above, it is preferred that the fragments or variants thereof comprise:—

(A) an amino acid sequence encoded by a nucleic acid sequence, wherein said nucleic acid sequence has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% nucleic acid sequence identity with SEQ ID NO:1;

(B) an amino acid sequence encoded by a nucleic acid sequence, wherein said nucleic acid sequence is hybridizable under stringent conditions to a nucleic acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% nucleic acid sequence identity with SEQ ID NO:1; or

(C) an amino acid sequence encoded by a nucleic acid sequence, wherein said nucleic acid sequence is complementary to a nucleic acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% nucleic acid sequence identity with SEQ ID NO:1.

Preferably, the fragments thereof comprise (i) at least four, preferably at least five, preferably at least six, preferably at least seven, preferably at least eight consecutive amino acids from SEQ ID NO:2 or (ii) a fragment of the nucleic acid sequence of SEQ ID NO:1 which encodes at least four, preferably at least five, preferably at least six, preferably at least seven, preferably at least eight consecutive amino acids from SEQ ID NO:2. Longer fragments are also preferred, for example at least about 10, 15, 20, 25, 30, 50, 75, 100, 150, 200, 225 and up to at least about 250 amino acids of SEQ ID NO:2 or corresponding coding fragments of SEQ ID NO:1. Fragments may also include truncated peptides that have x amino acids deleted from the N-terminus and/or C-terminus. In such truncations, x may be 1 or more (i.e. 1, 2, 3, 4, 5, 6, 7, 8, 9, 10, 15, 20, 30, 40, 50, 60, 70, 80, 90, 100 or more), but preferably less than 150 amino acids of SEQ ID NO:2 or corresponding coding fragments of SEQ ID NO:1.

Preferably, the fragments or variants thereof are functional fragments or variants thereof.

According to another aspect of the present invention, there is provided a method for diagnosing gastrointestinal cancer in a patient or for identifying a patient at risk of developing gastrointestinal cancer, the method comprising:

(a) determining an amount of the cancer specific biomarker in a sample obtained from a patient;

(b) comparing the amount of the determined cancer specific biomarker in the sample from the patient to the amount of the cancer specific biomarker in a normal control; wherein a difference in the amount of the cancer specific biomarker in the sample from the patient compared to the amount of the cancer specific biomarker in the normal control is associated with the presence of gastrointestinal cancer or is associated with a risk of developing gastrointestinal cancer, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

According to another aspect of the present invention, there is provided a method for monitoring the progression of gastrointestinal cancer in a patient, the method comprising:

(a) determining an amount of the cancer specific biomarker in a sample obtained from a patient;

(b) comparing the amount of the determined cancer specific biomarker in the sample from the patient to the amount of the cancer specific biomarker in a normal control; and

(c) repeating steps (a) and (b) at two or more time intervals, wherein an increase in the amount of the cancer specific biomarker from the patient over time is associated with an increase in the progression of gastrointestinal cancer and a decrease in the amount of the cancer specific biomarker from the patient over time is associated with a decrease in the progression of gastrointestinal cancer, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Accordingly, the methods of the present invention can be used to detect the onset, progression, stabilisation, amelioration and/or remission of gastrointestinal cancer.

Preferably, the control may be from the same patient from a previous sample, to thus monitor onset or progression. However, it is also preferred that the control may be normalised for a population, particularly a healthy or normal population, where there is no gastrointestinal cancer. In other words, the control may consist of the level of a biomarker found in a normal control sample from a normal subject.

Accordingly, in one example of the present invention, there is provided a method of diagnosing or monitoring the progression of gastrointestinal cancer, comprising detecting and/or quantifying the cancer specific biomarker in a biological fluid obtained from a patient, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

As discussed above, it is preferred that at least two detection and/or quantification steps are provided, spaced apart temporally.

Preferably, the steps are spaced apart by a few days, weeks, years or months, to determine whether the levels of the cancer specific biomarker have changed, thus indicating whether there has been a change in the progression of the cancer, enabling comparisons to be made between a level of the biomarker in samples taken on two or more occasions, as an increase in the level of the biomarker over time is indicative of the onset or progression of the cancer, whereas a decrease in the level of the biomarker may indicate amelioration and/or remission of the cancer.

Preferably, the difference in the level of the biomarker is statistically significant, determined by using a “t-test” providing confidence intervals of preferably at least about 80%, preferably at least about 85%, preferably at least about 90%, preferably at least about 95%, preferably at least about 99%, preferably at least about 99.5%, preferably at least about 99.95%, preferably at least about 99.99%.

The biomarkers and methods of the invention are particularly useful in detecting early stage cancer and are more sensitive than known methods for detecting early stage gastrointestinal cancer. Thus, the biomarkers and methods of the invention are particularly useful for confirming cancer when a patient has tested negative for cancer using conventional methods.

Prognosis and choice of treatment are dependent upon the stage of the cancer and the patient's general state of health.

In relation to oesophageal cancer, patients with Stage 0 cancer have carcinoma in situ, which is characterized by cancer cells that involve only the superficial layer of cells lining the eosophagus. Patients with Stage I cancer have cancer that invades beneath the surface lining of the eosophagus, but not into the muscle wall of the eosophagus, the lymph nodes or other locations in the body. Patients with Stage II cancer have cancer that invades into or through the muscular wall of the eosophagus, but not into nearby local structures (Stage IIA). When there is regional lymph node involvement with any extent of primary cancer but no invasion of local structures, this is called Stage IIB. Patients with Stage III cancer have cancer that invades through the wall of the eosophagus and has spread to the lymph nodes and/or invaded adjacent structures. Patients with Stage IV cancer have metastatic cancer that has spread to distant sites.

In relation to gall bladder cancer, in Stage 0 cancer, abnormal cells are found in the innermost (mucosal) layer of the gallbladder. These abnormal cells may become cancer and spread into nearby normal tissue. Stage I cancer is divided into Stages IA and IB. In Stage IA, cancer has spread beyond the innermost (mucosal) layer to the connective tissue or to the muscle layer. In stage IB, cancer has spread beyond the muscle layer to the connective tissue around the muscle. Stage II cancer is divided into Stages IIA and IIB. In Stage HA, cancer has spread beyond the tissue that covers the gallbladder and/or to the liver and/or to one nearby organ, such as the stomach, small intestine, colon, pancreas, or bile ducts outside the liver. In Stage IIB, cancer has spread in one of the following ways: (1) beyond the innermost layer to the connective tissue and to nearby lymph nodes; or (2) to the muscle layer and nearby lymph nodes; or (3) beyond the muscle layer to the connective tissue around the muscle and nearby lymph nodes; or (4) through the tissue that covers the gallbladder and/or to the liver and/or to one nearby organ, such as the stomach, small intestine, colon, pancreas, or bile ducts outside the liver, and to nearby lymph nodes. In Stage III, cancer has spread to a main blood vessel in the liver or to nearby organs and may have spread to nearby lymph nodes. In Stage IV, cancer has spread to nearby lymph nodes and/or to organs far away from the gallbladder.

In relation to stomach cancer, patients with Stage 0 cancer have what is referred to as carcinoma in situ, which is cancer that involves only the superficial layer of cells lining the stomach. Patients with Stage I cancer have cancer that invades beneath the surface layer of cells lining the stomach, but not into the muscle wall of the stomach. When there is no lymph node involvement or distant spread of cancer, the cancer is referred to as Stage IA cancer. When the cancer invades beneath the surface layer of cells and has spread to 1-6 lymph nodes or invades into the muscle of the wall of the stomach without regional lymph node or distant spread, it is referred to as Stage IB cancer. Patients with Stage II cancer have cancer that invades into or through the muscular wall of the stomach, but not into nearby local structures or have regional lymph node involvement with any extent of primary cancer, but no invasion of local structures. Patients with Stage III cancer have spread of cancer to structures adjacent to the stomach and/or to regional lymph nodes. Stage III cancer can be further divided into Stage IIIA and Stage IIB. Stage IIIA cancer either 1) invades into the muscle of the wall of the stomach with 7-15 lymph nodes involved, or 2) invades the lining of the abdomen (peritoneum) without invading local structures with 1-6 lymph nodes involved, or 3) invades the adjacent local structures without lymph node spread. Stage IIIB cancer invades the lining of the abdomen (peritoneum) with 7-15 lymph nodes involved. Patients with Stage IV gastric cancer have cancer that invades adjacent structures and lymph nodes or has spread to distant sites.

In relation to liver cancer, Stage I cancer is found in one location of the liver and could be treated surgically. Stage II cancer is found in one or more locations in the liver and may be treated surgically. Stage III cancer has spread to more than one location in the liver and/or to other parts of the body. Stage IV cancer involves multiple sites throughout the body.

In relation to pancreatic cancer, Stage I cancer, refers to cancer that is confined to the pancreas. Stage II cancer has spread to the duodenum, bile ducts, or fat surrounding the pancreas, but does not invade any local lymph nodes and cannot be detected in other locations in the body. Stage III cancer has spread to local lymph nodes and major blood vessels. Stage IV cancer has spread to distant locations in the body, such as the liver, lungs, or adjacent organs including the stomach, spleen, and/or the bowel.

In relation to bile duct cancer, Stage IA cancer is contained within the bile duct. Stage IB cancer has spread through the wall of the bile duct but has not spread into nearby lymph nodes or other structures. Stage IIA cancer has spread into the liver, pancreas or gall bladder or to the nearby blood vessels, but not the lymph nodes. Stage IIB cancer has spread into nearby lymph nodes. Stage III cancer is affecting the main blood vessels that take blood to and from the liver, or it has spread into the small or large bowel, the stomach or the abdominal wall. Lymph nodes in the abdomen may also be affected. Stage IV cancer has spread to distant parts of the body such as the lungs.

In relation to small intestine cancer, Stage I cancer is contained within the small bowel lining or spread into the muscle wall. Stage II cancer has spread through the muscle wall and possibly to nearby organs. Stage III cancer has spread to nearby lymph nodes. Stage IV cancer has spread to lymph nodes and other parts of the body.

In relation to anal cancer, Stage I cancer only affects the anus and is smaller than 2 cm in size. It has not begun to spread into the sphincter muscle. Stage II cancer is larger than 2 cm in size, but has not spread into nearby lymph nodes or to other parts of the body. Stage IIIA cancer has spread to the lymph nodes close to the rectum, or to nearby organs such as the bladder or vagina. Stage IIIB cancer has either spread to the lymph nodes in the groin and pelvis, or to the lymph nodes close to the anus, as well as nearby organs such as the bladder or vagina. Stage IV cancer has spread to lymph nodes in the abdomen or to other parts of the body, such as the liver.

In relation to colon cancer, Stage I cancer does not penetrate the wall of the colon into the abdominal cavity, has not spread to any adjacent organs or local lymph nodes and cannot be detected in other locations in the body. Stage II cancer has penetrated the wall of the colon into the abdominal cavity, but does not invade any of the local lymph nodes and cannot be detected in other locations in the body. Stage III cancer has penetrated the wall of the colon into the abdominal cavity and invaded any of the local lymph nodes, but cannot be detected in other locations in the body. Stage IV cancer has spread to distant locations in the body; this may include the liver, lungs, bones, distant lymph nodes or other sites.

In relation to rectal cancer, Stage I cancer is confined to the lining of the rectum. Stage II cancer has penetrated the wall of the rectum, but does not invade any of the local lymph nodes and cannot be detected in other locations in the body. Stage III cancer has penetrated the wall of the rectum and invaded one or more local lymph nodes, but cannot be detected in other locations in the body. Stage IV cancer has spread to distant locations in the body, which may include the liver, lungs, bones or other sites.

It will be appreciated that the term “early stage” as used herein can be said to refer to stage 0, stage I and/or stage II, as discussed above.

With regard to the term “late stage” as used herein, it will be appreciated that this term can be said to refer to stage III and/or stage IV.

It will be appreciated that the “early stage” and “late stage” nature of the cancer disease states can be determined by a physician. It is also envisaged that they may be associated with non-metastatic and metastatic states, respectively.

In one aspect, there are provided methods according to the present invention for detecting early stage cancer, wherein an increase between the control and the sample obtained from the patient is indicative of early stage cancer. Preferably, the increase is at least about 100%, preferably at least about 125%, preferably at least about 150%, preferably at least about 200%, preferably at least about 250%, preferably at least about 300%, preferably at least about 500%.

Also provided are methods according to the present invention for detecting late stage cancer wherein an increase between the control and the sample obtained from the patient is indicative of late stage cancer. Preferably, the increase is at least about 100%, preferably at least about 125%, preferably at least about 150%, preferably at least about 200%, preferably at least about 250%, preferably at least about 300%, preferably at least about 500%, preferably at least about 750%, preferably at least about 1000%.

Further provided are methods according to the present invention for monitoring a change in stage of cancer, wherein an increase, relative to an earlier stage sample or control is indicative of progression of the cancer from an earlier stage to later stage of disease, for example from stage 0 to stage I, from stage I to stage II, from stage II to stage III, from stage III to stage IV, from early stage to late stage, or from stages in between, for example from stage WA to stage IVB in accordance with cancer specific stages described above. Preferably, the increase is at least about 100%, preferably at least about 125%, preferably at least about 150%, preferably at least about 200%, preferably at least about 250%, preferably at least about 300%, preferably at least about 500%, preferably at least about 750%, preferably at least about 1000%.

It is preferred that the gastrointestinal cancer specific biomarker is indicative of the presence of gastrointestinal cancer or the risk of developing gastrointestinal cancer when present at a level of at least about 2-fold, preferably at least about 3-fold, preferably at least about 4-fold, preferably at least about 5-fold, preferably at least about 10-fold, preferably at least about 20-fold, preferably at least about 30-fold, preferably at least about 40-fold, preferably at least about 50-fold, preferably at least about 75-fold, preferably at least about 100-fold that of a normal control.

Also provided by the present invention is a method for monitoring the efficacy of a treatment for gastrointestinal cancer, comprising detecting and/or quantifying the presence of the cancer specific biomarker in a biological sample obtained from a patient, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Preferably, in the methods of the present invention, detection and/or quantification of the cancer specific biomarker is by one or more of MALDI-TOF, SELDI, via interaction with a ligand or ligands, 1-D or 2-D gel-based analysis systems, Liquid Chromatography, combined liquid chromatography and Mass spectrometry techniques including ICAT(R) or iTRAQ(R), thin-layer chromatography, NMR spectroscopy, sandwich immunoassays, enzyme linked immunosorbent assays (ELISAs), radioimmunoassays (RAI), enzyme immunoassays (EIA), lateral flow/immunochromatographic strip tests, Western Blotting, immunoprecipitation, and particle-based immunoassays including using gold, silver, or latex particles, magnetic particles or Q-dots and immunohistochemistry on tissue sections.

Preferably, detection and/or quantification of the cancer specific biomarker is performed on a microtitre plate, strip format, array or on a chip.

Preferably, detection and/or quantification of the cancer specific biomarker is by an ELISA comprising antibodies specific for the cancer specific biomarker, preferably linked to a reporter.

Preferably, detection and/or quantification of the cancer specific biomarker is by a biosensor.

Preferably, the sample comprises biological fluid or tissue obtained from the patient. Preferably, the biological fluid or tissue comprises cellular fluid, ascites, urine, faeces, pancreatic fluid, fluid obtained during endoscopy blood or saliva. In preferred embodiments, the sample comprises, faeces, fluid obtained during endoscopy, blood or pancreatic fluid obtained from a patient.

It is also preferred that the biological fluid is substantially or completely free of whole/intact cells. Preferably the biological fluid is free of platelets and cell debris (such as that produced upon the lysis of cells). Preferably the biological fluid is free of both prokaryotic and eukaryotic cells.

Such samples can be obtained by any number of means known in the art, such as will be apparent to the skilled person. For instance, urine and faecal samples are easily attainable, whilst blood, ascites, serum or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration.

Although it is generally preferred that no invasive techniques are used to obtain the sample, it still may be preferable to obtain samples such as tissue homogenates, tissue sections and biopsy specimens.

Another aspect of the present invention relates to a method for treating a patient with gastrointestinal cancer, the method comprising administering to a patient a therapeutically effective amount of (i) a biomarker of the present invention or (ii) an antibody or fragment thereof that specifically binds to a biomarker of the present invention, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Another aspect of the present invention relates to a method for imaging gastrointestinal cancer in a patient, the method comprising administering to a patient an antibody or fragment thereof that specifically binds to a biomarker of the present invention, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Preferably, the antibody is conjugated to a detectable marker, for example a fluorescent marker or tag. Preferably, the antibody is a monoclonal antibody. Preferably, the antibody is conjugated to a growth inhibitory agent. Preferably, the antibody is conjugated to a cytotoxic agent, for example a toxin (e.g. an immunotoxin), antibiotic, lytic enzyme or radioactive isotope.

Another aspect of the present invention relates to a composition comprising a biomarker of the present invention or an antibody or fragment thereof that binds to a biomarker of the present invention.

Preferably, the composition is a pharmaceutical composition.

Also provided by the present invention is a vaccine comprising a biomarker of the present invention or an antibody or fragment thereof that binds to a biomarker of the present invention.

Another aspect of the present invention relates to use of the cancer specific biomarker, detectable in a body fluid, as a biomarker for gastrointestinal cancer, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Preferably, said use is in a method selected from the group consisting of: clinical screening, methods of prognosis assessment, monitoring the results of therapy, method to identify patients most likely to respond to a particular therapeutic treatment, and drug screening and development.

Another aspect of the present invention relates to use of (i) a biomarker of the present invention, or (ii) an antibody or fragment thereof that specifically binds to a biomarker of the present invention, in the manufacture of a medicament for the treatment of gastrointestinal cancer, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Also provided is a composition comprising (i) a biomarker of the present invention, or (ii) an antibody or fragment thereof that specifically binds to a biomarker of the present invention, wherein the composition is for use in the treatment of gastrointestinal cancer, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Another aspect of the present invention relates to an antibody or fragment thereof that specifically binds to a biomarker of the present invention for use in a method of imaging gastrointestinal cancer in a patient, optionally wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

In preferred embodiments, the methods and compositions of the invention are for treatment or diagnosis of disease at an early stage, for example, before symptoms of the disease appear.

In some embodiments, the methods and compositions of the invention are for treatment or diagnosis of disease at a clinical stage

According to another aspect of the present invention, there is provided a kit for use in the methods or uses described above, wherein the kit comprises a ligand capable of binding or specifically recognising the cancer specific biomarker, detectable in a body fluid and reporter means.

Preferably, the kit is an array or chip.

Preferably, the kit comprises a microtitre plate, test strip, array or chip.

### DETAILED DESCRIPTION OF THE INVENTION

Example embodiments of the present invention will now be described with reference to the accompanying figures.

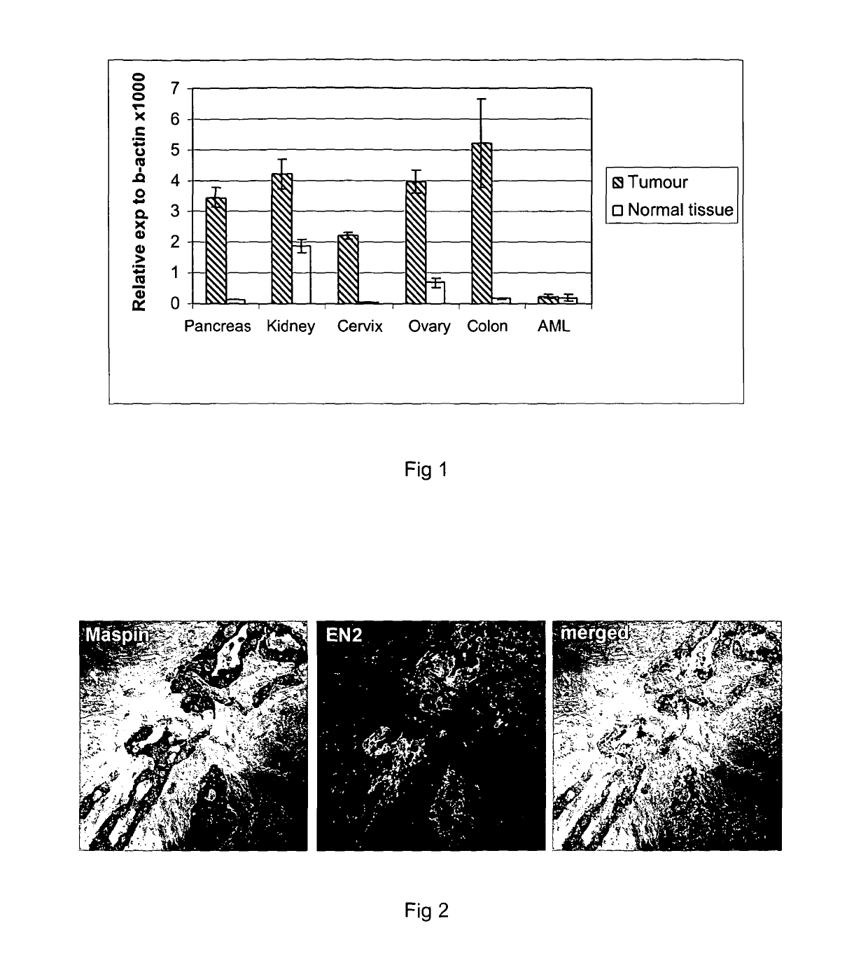

FIG. 1 shows the results of the experiments performed in Example 1 below;

FIG. 2 shows the results of the experiments performed in Example 2 below;

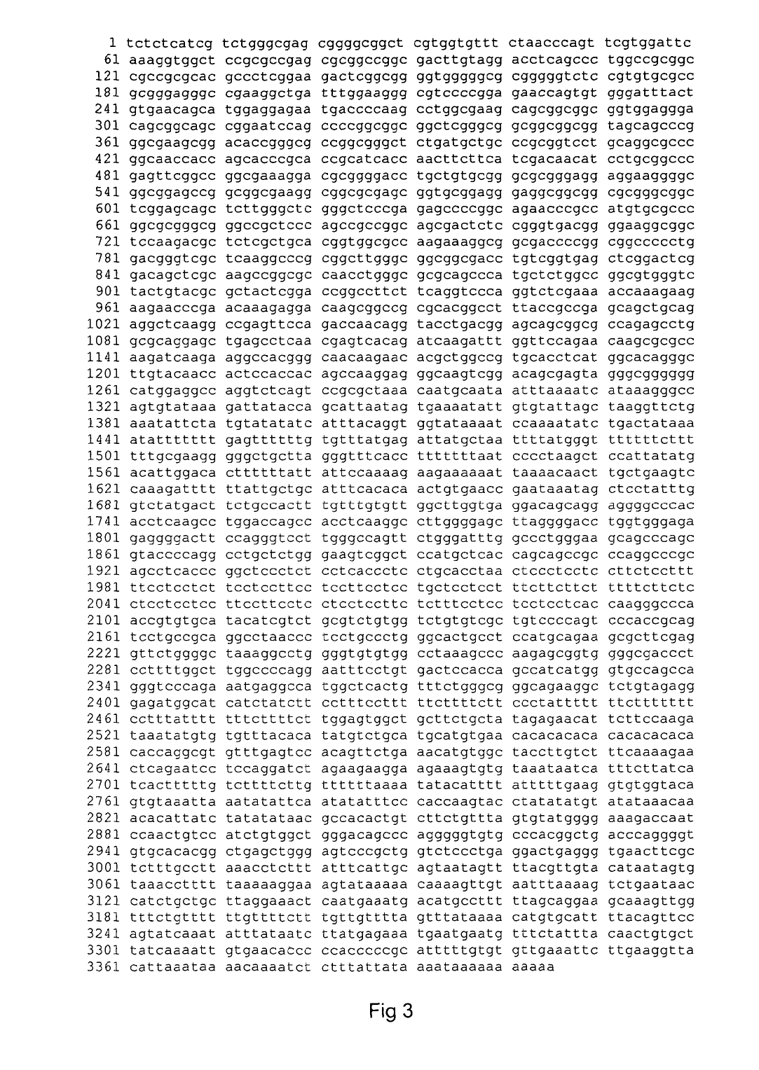

FIG. 3 shows the nucleic acid sequence of EN2 (SEQ ID NO:1); and

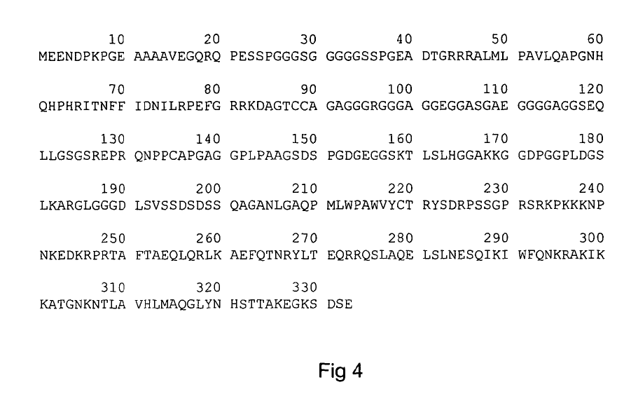

FIG. 4 shows the amino acid sequence of EN2 (SEQ ID NO:2).

The invention relates to gastrointestinal cancer specific biomarkers, preferably, wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

Within this specification, the terms “comprises” and “comprising” are interpreted to mean “includes, among other things”. These terms are not intended to be construed as “consists of only”.

Within this specification, the term “about” means plus or minus 20%, more preferably plus or minus 10%, even more preferably plus or minus 5%, most preferably plus or minus 2%.

As used herein, the term “therapeutically effective amount” means the amount of a composition which is required to reduce the severity of and/or ameliorate at least one condition or symptom which results from the disease in question.

Within this specification embodiments have been described in a way which enables a clear and concise specification to be written, but it is intended and will be appreciated that embodiments may be variously combined or separated without parting from the invention. For example, it will be appreciated that in all instances of gastrointestinal cancer, a preferred feature is wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

For clinical use, a compound according to the present invention or prodrug form thereof is formulated into a pharmaceutical formulation which is formulated to be compatible with its intended route of administration, for example for oral, rectal, parenteral or other modes of administration. Pharmaceutical formulations are usually prepared by mixing the active substance with a conventional pharmaceutically acceptable diluent or carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Examples of pharmaceutically acceptable diluents or carrier are water, gelatin, gum arabicum, lactose, microcrystalline cellulose, starch, sodium starch glycolate, calcium hydrogen phosphate, magnesium stearate, talcum, colloidal silicon dioxide, and the like. The use of such media and agents for pharmaceutically active substances is well known in the art. Except insofar as any conventional media or agent is incompatible with the active compound, use thereof in the compositions is contemplated.

Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, ‘chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Within this specification, “identity,” as it is known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. Percentage identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), all of which are incorporated herein by reference in their entirety. Preferred methods to determine identity are designed to give the largest match between the sequences tested. Methods to determine identity are codified in publicly available computer programs. Preferred computer program methods to determine percentage identity between two sequences include, but are not limited to, the GCG program package (Devereux, J., et al., Nucleic Acids Research 12(1): 387 (1984), which is incorporated herein by reference in its entirety), BLASTP, BLASTN, and FASTA (Atschul, S. F. et al., J. Molec. Biol. 215: 403-410 (1990), which is incorporated herein by reference in its entirety). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), which is incorporated herein by reference in its entirety). As an illustration, by a polynucleotide having a nucleotide sequence having at least, for example, 95% “identity” to a reference nucleotide sequence of “SEQ ID NO: A” it is intended that the nucleotide sequence of the polynucleotide is identical to the reference sequence except that the polynucleotide sequence may include up to five point mutations per each 100 nucleotides of the reference nucleotide sequence of “SEQ ID NO: A.” In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid sequence of “SEQ ID NO:B” is intended that the amino acid sequence of the polypeptide is identical to the reference sequence except that the polypeptide sequence may include up to five amino acid alterations per each 100 amino acids of the reference amino acid of “SEQ ID NO: B.” In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “hybridizes under stringent conditions” is intended to describe conditions for hybridization and washing under which nucleotide sequences encoding a receptor at least 50% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6. 3.1-6.3.6, which is incorporated herein by reference in its entirety. One example of stringent hybridization conditions are hybridization in 6× sodium chloride/sodium citrate (SSC) at about 45° C., followed by one or more washes in 0.2×SSC, 0.1% SDS at 50-65° C. In one embodiment, an isolated receptor nucleic acid molecule that hybridizes under stringent conditions to the sequence of SEQ ID NO:1 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Within this specification, “antibody or antibody fragment” refers to an antibody (for example IgG, IgM, IgA, IgD or IgE) or fragment (such as a Fab, F(ab′)2, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

Within this specification, the term “treatment” means treatment of an existing disease and/or prophylactic treatment in order to prevent incidence of a disease. As such, the methods of the invention can be used for the treatment, prevention, inhibition of progression or delay in the onset of disease.

The term “biomarker” is used throughout the art and means a distinctive biological or biologically-derived indicator of a process, event or condition. In other words, a biomarker is indicative of a certain biological state, such as the presence of cancerous tissue. In some cases, different forms of biomarkers can be indicative of certain disease states but, without being bound by theory, it is thought that merely the presence of elevated levels of the biomarkers of the present invention in body fluids such as ascites, is indicative of gastrointestinal cancer. Although it is not currently envisaged that different glycoforms, for instance, of the EN2 peptide, are secreted, these are nevertheless encompassed by the present invention. For instance, different glycoforms, such as altered glycoform structure or sugar content, may yet be determined for EN2, but these are encompassed and may even also be indicative of the progress of gastrointestinal cancer. Truncations, mutations, or deletions of or ligations to, the EN2 peptide, or fragment thereof, are also envisaged.

As discussed above, it has surprisingly been found that there is a significant increase in expression of the EN2 gene in gastrointestinal tumours compared to normal tissue. Furthermore, EN2 is found in the ascites of patients with gastrointestinal cancer. It is thought that EN-2 may be secreted or may be detectable in body fluids due to leaking from damaged or dead cells. Such increased levels are indicative of both early stage and late stage gastrointestinal cancer. Whilst there is a significant rise between control or normal levels and early stage gastrointestinal cancer, there is also a very significant increase between early and late stage gastrointestinal cancer. Broadly, it is an advantage of the present invention that the substance and also the state of the cancer can be detected. This aids in the prognosis and provision of suitable therapies.

It is another advantage of the present invention that an accurate diagnosis can be provided without resorting to unpleasant and potentially harmful invasive procedures, which may also be inaccurate. Furthermore, the present invention is particularly sensitive. Preferably the methods of the present invention may detect the onset of cancer prior to any other detection method and prior to the onset of the overt symptoms of cancer. Thus, the cancer may be treated at an early stage when it is more susceptible to such treatment and less likely to have entered the metastatic stage.

The biomarkers of the present invention can be used in methods of diagnosis, for instance clinical screening, and in methods of prognosis assessment, monitoring the results of therapy, identifying patients most likely to respond to a particular therapeutic treatment, drug screening and development. Furthermore, the biomarkers of the present invention and uses thereof are valuable for identification of new drug treatments and for discovery of new targets for drug treatment.

The term “diagnosis” encompasses identification, confirmation, and or characterisation of the presence or absence of gastrointestinal cancer, together with the developmental stage thereof, such as early stage or late stage, or benign or metastatic cancer.

### EXAMPLES

### Example 1

We have studied the expression of the EN2 gene using RT-PCR from whole RNA extracted from pancreatic, renal, ovarian, cervical and colorectal tumours, and in normal tissue from the relevant organs and tissues. The RNA was obtained from Ambion Inc, USA, now part of Invitrogen Ltd. The product codes for these RNAs are:

Pancreas—tumour: AM7229, normal tissue: AM7954

Kidney—tumour/normal tissue set: AM7252

Cervix—tumour/normal tissue set: AM7276

Ovary—tumour/normal tissue set: AM7256

Colorectal cancer—tumour/normal tissue set: AM7236

In addition we also extracted whole RNA from the acute myelogenous leukaemia (AML) derived cell line KG-1 and from peripheral blood mononuclear cells (PBMCs) donated by healthy volunteers.

RT-PCR method

RNA extraction was performed using the RNeasy mini kit (Qiagen, Crawley, UK) following the manufacturers instructions. RNA was first denatured by heating to 65° C. for five minutes. 1 μg of RNA was incubated in a volume of 50 μl at 37° C. for one hour with final concentrations of 10 mM DTT, 1 mM dNTP mix, as well as 100 ng/ml polyT primers, 200 units of reverse transcriptase (Invitrogen, USA) and 40 units of RNaseOUT (Invitrogen, USA). The cDNA synthesis reaction was terminated by placing tubes at 80° C. for five minutes. RT-PCR was performed using the Stratagene MX4000 Real Time PCR machine, measuring PCR product accumulation during the exponential phase of the reaction by SYBR green fluorescence. The expression of EN2 was calculated relative to that of the Beta-actin gene, the expression of which is relatively constant in many cell types.

### QPCR Primer Sequences:

| Beta-actin (human): | |
|---|---|
| (SEQ ID NO: 3) | |
| HsBeta-ActinF: | 5′ ATGTACCCTGGCATTGCCGAC 3′ |
| (SEQ ID NO: 4) | |
| HsBeta-ActinR: | 5′ GACTCGTCATACTCCTGCTTG 3′ |
| EN2 (human): | |
| (SEQ ID NO: 5) | |
| HsEN2F: | 5′ GAACCCGAACAAAGAGGACA 3′ |
| (SEQ ID NO: 6) | |
| HsEN2R: | 5′ CGCTTGTTCTGGAACCAAAT 3′ |

### EN2 Expression in Tumours and Normal Tissue

Results of the RT-PCR are summarised in FIG. 1. The error bars represent standard deviation (n=5). P values for significant differences in values: * −p<0.05, ** −p<0.01. The results reveal that EN2 is strongly expressed compared to normal cells for all of the cancers except AML.

### Example 2

### Immunohistochemical (ICH) Detection of EN2 Protein in Pancreatic Cancer

In addition to the results shown in Example 1, we have also shown that EN2 protein is present in pancreatic tumours but not in the surrounding normal tissue. This was achieved by comparing the staining of a known pancreatic cancer marker, Maspin, to EN2 in pancreatic tumour sections, revealing a very high degree of overlap, with minimal EN2 staining in non-tumour cells (FIG. 2).

FIG. 2 shows a section through a pancreatic tumour. Tumour cells were stained using the pancreatic cancer specific antigen (Maspin). The same section was also stained with fluorescently labelled anti-EN2. The merged image reveals an almost identical pattern of staining. Magnification: x60.

The ICH method was performed as follows.

### Pancreatic Tumour Sections—Enzymatic Staining for EN2

1. Deparaffinze slides in three changes of 100% xylene for 5 minutes each
2. Wash twice in 100% ethanol
3. 20 mins in 0.3% Methanol/H202 (300 mls methanol+900 ul H2O2)
4. Rehydrate in an ethanol series of 70% and 50%
5. Rinse slides in distilled water for 5 mins
6. Incubate slides in boiling citrate buffer pH6.0 for 12 mins*
7. Leave the slides to cool down for 2 hours
8. Wash in distilled water for 3 mins
9. 2 washes of 3 mins in PBS
10. Incubate sections for 15 minutes with 2.4% horse serum in PBS/BSA 1% in a moist chamber. (Blocking serum needs to be raised in the same species as the 2° Ab).
11. Incubate overnight with 1° antibody (Abcam goat-anti EN2) in moist chamber, room temp
12. 3 washes of 3 mins in PBS.
13. Incubate sections for 30 minutes with diluted biotinylated “universal” secondary antibody.
14. 3 washes of 3 mins in PBS
15. Incubate sections for 30 minutes with VECTASTAIN R.T.U. ABC Reagent.
16. 3 washes of 3 mins in PBS
17. Incubate sections in peroxidase substrate solution until desired stain intensity develops
18. (10 mins for each slide, using impact DAB solution)
19. Rinse sections in distilled water.
20. Counterstain, (Haematoxylin QS: 100 μl/slide for max 45 secs) and put slides back in running tap water
21. Dehydrated slide in series of alcohol (50%, 70%, 100%, Xylene 1, 2, 3, quick dips for each.
22. Mounted slides with VectaMount mounting medium and stored slides at room temp
*Microwave Antigen Retrieval with 0.01M Citrate Buffer pH6.0
1. Prepare 0.01M citrate buffer from stock solution: 1:10 dilution with distilled water
2. Measure pH and bring to pH6.0 using 0.1M citric acid
3. Pour 1 L of citrate buffer into plastic container and microwave for 20 minutes High
4. Add sections in a rack to the boiling citrate buffer and microwave for a further 12 minutes

### Making 0.3% H202:

### 300 ml Methanol

### 900 μl H2O2 (30%) Hydrogen Peroxidase

Making Peroxidase Substrate Serum from imPACT DAB:

### 1 ml Diluent and 1 Drop Chromogen

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

1. A gastrointestinal cancer specific biomarker comprising:—
(i) a nucleic acid sequence comprising SEQ ID NO: 1, or a fragment or variant thereof, or a nucleic acid molecule which comprises said nucleic acid sequence; or
(ii) an amino acid sequence comprising SEQ ID NO:2, or a fragment or variant thereof, or an amino acid molecule which comprises said amino acid sequence.

2. The biomarker according to claim 1, wherein the fragments or variants thereof comprise:—
(i) a nucleic acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% nucleic acid sequence identity with SEQ ID NO:1, a nucleic acid sequence that is hybridizable thereto under stringent conditions, and/or a nucleic acid sequence that is complementary thereto;
(ii) an amino acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% amino acid sequence identity with SEQ ID NO:2, or
(iii) an amino acid sequence encoded by a nucleic acid sequence of (i).

3. The biomarker according to claim 1, wherein the fragments thereof comprise (i) at least four, preferably at least five, preferably at least six, preferably at least seven, preferably at least eight consecutive amino acids from SEQ ID NO:2 or (ii) a fragment of the nucleic acid sequence of SEQ ID NO: 1 which encodes at least four, preferably at least five, preferably at least six, preferably at least seven, preferably at least eight consecutive amino acids from SEQ ID NO:2.

4. The biomarker according to claim 1, wherein the fragments or variants thereof are functional fragments or variants thereof.

5. The biomarker according to claim 1, wherein the biomarker is selected from an oesophageal cancer specific biomarker, a gall bladder cancer specific biomarker, a stomach cancer specific biomarker, a liver cancer specific biomarker, a pancreatic cancer specific biomarker, a bile duct cancer specific biomarker, a small intestine cancer specific biomarker, a colorectal cancer specific biomarker and an anal cancer specific biomarker, optionally, wherein the colorectal cancer specific biomarker is selected from a colon cancer specific biomarker and a rectal cancer specific biomarker.

6. A method for diagnosing gastrointestinal cancer in a patient or for identifying a patient at risk of developing gastrointestinal cancer, the method comprising:
(a) determining an amount of a cancer specific biomarker according to claim 1 in a sample obtained from a patient;
(b) comparing the amount of the determined cancer specific biomarker in the sample from the patient to the amount of the cancer specific biomarker in a normal control; wherein a difference in the amount of the cancer specific biomarker in the sample from the patient compared to the amount of the cancer specific biomarker in the normal control is associated with the presence of gastrointestinal cancer or is associated with a risk of developing gastrointestinal cancer.

7. The method according to claim 6 for detecting early stage cancer, wherein an increase between the control and the sample obtained from the patient is indicative of early stage cancer.

8. The method according to claim 6 for detecting late stage cancer wherein an increase between the control and the sample obtained from the patient is indicative of late stage cancer.

9. A method for monitoring the progression of gastrointestinal cancer in a patient, the method comprising:
(a) determining an amount of a cancer specific biomarker according to claim 1 in a sample obtained from a patient;
(b) comparing the amount of the determined cancer specific biomarker in the sample from the patient to the amount of the cancer specific biomarker in a normal control; and
(c) repeating steps (a) and (b) at two or more time intervals, wherein an increase in the amount of the cancer specific biomarker from the patient over time is associated with an increase in the progression of gastrointestinal cancer and a decrease in the amount of the cancer specific biomarker from the patient over time is associated with a decrease in the progression of gastrointestinal cancer.

10. The method according to claim 9 for monitoring a change in stage of cancer, wherein an increase, relative to an earlier stage sample or control is indicative of progression of the cancer from an earlier stage to later stage of disease.

11. A method for monitoring the efficacy of a treatment for gastrointestinal cancer, comprising detecting and/or quantifying the presence of a cancer specific biomarker according to claim 1 in a sample obtained from a patient.

12. The method according to claim 6, wherein the sample comprises biological fluid or tissue obtained from the patient.

13. The method according to claim 12, wherein the biological fluid or tissue comprises blood, urine, fluid obtained during endoscopy, pancreatic fluid or faeces.

14. A method for treating a patient with gastrointestinal cancer, the method comprising administering to a patient a therapeutically effective amount of (i) a biomarker according to claim 1, or (ii) an antibody or fragment thereof that specifically binds to a biomarker according to claim 1.

15. The method according to claim 14, wherein the antibody is conjugated to a cytotoxic agent.

16. A method for imaging gastrointestinal cancer in a patient, the method comprising administering to a patient an antibody or fragment thereof that specifically binds to a biomarker according to claim 1.

17. The method according to claim 14, wherein the antibody is conjugated to a detectable marker.

18. The method according to claim 6, wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

19. An antibody or fragment thereof that binds to a biomarker according to claim 1.

20. A pharmaceutical composition comprising a composition according to claim 1.

21. A gastrointestinal cancer vaccine comprising a biomarker according to claim 1.

22. The gastrointestinal cancer vaccine according to claim 21, selected from an oesophageal cancer vaccine, a gall bladder cancer vaccine, a stomach cancer vaccine, a liver cancer vaccine, a pancreatic cancer vaccine, a bile duct cancer vaccine, a small intestine cancer vaccine, a colorectal cancer vaccine and an anal cancer vaccine, optionally, wherein the colorectal cancer vaccine is selected from a colon cancer vaccine and a rectal cancer vaccine.

23-29. (canceled)

30. A kit, wherein the kit comprises a ligand capable of binding or specifically recognising a cancer specific biomarker according to claim 1, detectable in a body fluid and reporter means.

31. The method according to claim 9, wherein the sample comprises biological fluid or tissue obtained from the patient.

32. The method according to claim 31, wherein the biological fluid or tissue comprises blood, urine, fluid obtained during endoscopy, pancreatic fluid or faeces.

33. The method according to claim 11, wherein the sample comprises biological fluid or tissue obtained from the patient.

34. The method according to claim 33, wherein the biological fluid or tissue comprises blood, urine, fluid obtained during endoscopy, pancreatic fluid or faeces.

35. The method according to claim 16, wherein the antibody is conjugated to a detectable marker.

36. The method according to claim 9, wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

37. The method according to claim 11, wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

38. The method according to claim 14, wherein the gastrointestinal cancer is selected from oesophageal cancer, gall bladder cancer, stomach cancer, liver cancer, pancreatic cancer, bile duct cancer, small intestine cancer, colorectal cancer and anal cancer, optionally, wherein the colorectal cancer is selected from colon cancer and rectal cancer.

39. A pharmaceutical composition comprising a composition according to claim 19.


SEQUENCE LISTING (6 sequences)

SEQ ID NO: 1 - DNA, 3405 bp, Homo sapiens
tctctcatcgtctgggcgagcggggcggctcgtggtgtttctaacccagttcgtggattcaaaggtggctccgcgccgagcgcggccggcgacttgtaggacctcagccctggccgcggccgccgcgcacgccctcggaagactcggcggggtgggggcgcgggggtctccgtgtgcgccgcgggagggccgaaggctgatttggaagggcgtccccggagaaccagtgtgggatttactgtgaacagcatggaggagaatgaccccaagcctggcgaagcagcggcggcggtggagggacagcggcagccggaatccagccccggcggcggctcgggcggcggcggcggtagcagcccgggcgaagcggacaccgggcgccggcgggctctgatgctgcccgcggtcctgcaggcgcccggcaaccaccagcacccgcaccgcatcaccaacttcttcatcgacaacatcctgcggcccgagttcggccggcgaaaggacgcggggacctgctgtgcgggcgcgggaggaggaaggggcggcggagccggcggcgaaggcggcgcgagcggtgcggagggaggcggcggcgcgggcggctcggagcagctcttgggctcgggctcccgagagccccggcagaacccgccatgtgcgcccggcgcgggcgggccgctcccagccgccggcagcgactctccgggtgacggggaaggcggctccaagacgctctcgctgcacggtggcgccaagaaaggcggcgaccccggcggccccctggacgggtcgctcaaggcccgcggcttgggcggcggcgacctgtcggtgagctcggactcggacagctcgcaagccggcgccaacctgggcgcgcagcccatgctctggccggcgtgggtctactgtacgcgctactcggaccggccttcttcaggtcccaggtctcgaaaaccaaagaagaagaacccgaacaaagaggacaagcggccgcgcacggcctttaccgccgagcagctgcagaggctcaaggccgagttccagaccaacaggtacctgacggagcagcggcgccagagcctggcgcaggagctgagcctcaacgagtcacagatcaagatttggttccagaacaagcgcgccaagatcaagaaggccacgggcaacaagaacacgctggccgtgcacctcatggcacagggcttgtacaaccactccaccacagccaaggagggcaagtcggacagcgagtagggcggggggcatggaggccaggtctcagtccgcgctaaacaatgcaataatttaaaatcataaagggccagtgtataaagattataccagcattaatagtgaaaatattgtgtattagctaaggttctgaaatattctatgtatatatcatttacaggtggtataaaatccaaaatatctgactataaaatatttttttgagttttttgtgtttatgagattatgctaattttatgggtttttttcttttttgcgaagggggctgcttagggtttcacctttttttaatcccctaagctccattatatgacattggacacttttttattattccaaaagaagaaaaaattaaaacaacttgctgaagtccaaagattttttattgctgcatttcacacaactgtgaaccgaataaatagctcctatttggtctatgacttctgccactttgtttgtgttggcttggtgaggacagcaggaggggcccacacctcaagcctggaccagccacctcaaggccttggggagcttaggggacctggtgggagagaggggacttccagggtccttgggccagttctgggatttggccctgggaagcagcccagcgtaccccaggcctgctctgggaagtcggctccatgctcaccagcagccgcccaggcccgcagcctcacccggctccctctcctcaccctcctgcacctaactccctcctccttctcctttttcctcctcttcctccttcctccttcctcctgctcctcctttcttcttctttttcttctcctcctcctccttccttcctcctcctccttctctttcctcctcctcctcaccaagggcccaaccgtgtgcatacatcgtctgcgtctgtggtctgtgtcgctgtccccagtcccaccgcagtcctgccgcaggcctaaccctcctgccctgggcactgcctccatgcagaagcgcttcgaggttctggggctaaaggcctggggtgtgtggcctaaagcccaagagcggtggggcgaccctccttttggcttggccccaggaatttcctgtgactccaccagccatcatgggtgccagccagggtcccagaaatgaggccatggctcactgtttctgggcgggcagaaggctctgtagagggagatggcatcatctatcttcctttcctttttcttttcttccctatttttttctttttttcctttatttttttcttttcttggagtggctgcttctgctatagagaacattcttccaagataaatatgtgtgtttacacatatgtctgcatgcatgtgaacacacacacacacacacacacaccaggcgtgtttgagtccacagttctgaaacatgtggctaccttgtctttcaaaagaactcagaatcctccaggatctagaagaaggaagaaagtgtgtaaataatcatttcttatcatcactttttgtcttttcttgttttttaaaatatacattttatttttgaaggtgtggtacagtgtaaattaaatatattcaatatatttcccaccaagtacctatatatgtatataaacaaacacattatctatatataacgccacactgtcttctgtttagtgtatggggaaagaccaatccaactgtccatctgtggctgggacagcccagggggtgtgcccacggctgacccaggggtgtgcacacggctgagctgggagtcccgctggtctccctgaggactgagggtgaacttcgctctttgccttaaacctctttatttcattgcagtaatagttttacgttgtacataatagtgtaaacctttttaaaaaggaaagtataaaaacaaaagttgtaatttaaaagtctgaataaccatctgctgcttaggaaactcaatgaaatgacatgcctttttagcaggaagcaaagttggtttctgttttttgttttctttgttgttttagtttataaaacatgtgcattttacagttccagtatcaaatatttataatcttatgagaaatgaatgaatgtttctatttacaactgtgcttatcaaaattgtgaacacccccacccccgcatttttgtgtgttgaaattcttgaaggttacattaaataaaacaaaatctctttattataaaataaaaaaaaaaa

SEQ ID NO: 2 - protein, 570 aa, Homo sapiens
MEENDPKPGEAAAAVEGQRQPESSPGGGSGGGGGSSPGEADTGRRRALMLPAVLQAPGNHQHPHRITNFFIDNILRPEFGRRKDAGTCCAGAGGGRGGGAGGEGGASGAEGGGGAGGSEQLLGSGSREPRQNPPCAPGAGGPLPAAGSDSPGDGEGGSKTLSLHGGAKKGGDPGGPLDGSLKARGLGGGDLSVSSDSDSSQAGANLGAQPMLWPAWVYCTRYSDRPSSGPRSRKPKKKNPNKEDKRPRTAFTAEQLQRLKAEFQTNRYLTEQRRQSLAQELSLNESQIKIWFQNKRAKIKKATGNKNTLAVHLMAQGLYNHSTTAKEGKSDSEGGGAGGEGGASGAEGGGGAGGSEQLLGSGSREPRQNPPCAPGAGGPLPAAGSDSPGDGEGGSKTLSLHGGAKKGGDPGGPLDGSLKARGLGGGDLSVSSDSDSSQAGANLGAQPMLWPAWVYCTRYSDRPSSGPRSRKPKKKNPNKEDKRPRTAFTAEQLQRLKAEFQTNRYLTEQRRQSLAQELSLNESQIKIWFQNKRAKIKKATGNKNTLAVHLMAQGLYNHSTTAKEGKSDSE

SEQ ID NO: 3 - DNA, 21 bp, Artificial Sequence
  QPCR Primer Sequence
atgtaccctggcattgccgac

SEQ ID NO: 4 - DNA, 21 bp, Artificial Sequence
  QPCR Primer Sequence
gactcgtcatactcctgcttg

SEQ ID NO: 5 - DNA, 20 bp, Artificial Sequence
  QPCR Primer Sequence
gaacccgaacaaagaggaca

SEQ ID NO: 6 - DNA, 20 bp, Artificial Sequence
  QPCR Primer Sequence
cgcttgttctggaaccaaat